(19) United States
(12) Patent Application Publication
(10) Pub. No.: US 2020/0323790 A1
(43) Pub. Date: Oct. 15, 2020

(54) METHOD FOR TREATMENT OF ACUTE LUNG INJURY BY USE OF KIRENOL

(71) Applicant: CHUNG SHAN MEDICAL UNIVERSITY, TAICHUNG CITY (TW)

(72) Inventors: YU-HSIANG KUAN, TAICHUNG CITY (TW); CHUN-JUNG CHEN, TAICHUNG CITY (TW); SHIUAN-SHINN LEE, TAICHUNG CITY (TW); YUNG-CHYUAN HO, TAICHUNG CITY (TW)

(21) Appl. No.: 16/382,662

(22) Filed: Apr. 12, 2019

(51) Int. Cl. A61K 31/047; A61P 11/00

(52) U.S. Cl. CPC A61K 31/047; A61P 11/00

(57) ABSTRACT

A method for treatment of acute lung injury by use of kirenol is revealed. The method includes a step of applying an effective dose of kirenol to an individual in need for reducing hyaline membrane formation, neutrophil infiltration, pulmonary edema, and pulmonary oxidative stress so as to treat acute lung injury.

### BACKGROUND OF THE INVENTION

### Field of the Invention

The present invention relates to a method for treatment of acute lung injury, especially to a method for treatment of symptoms related to acute lung injury by use of kirenol.

### Description of Related Art

Owing to air pollution that is getting worse, respiratory disease such as pneumonia, lung cancer, chronic obstructive pulmonary disease, asthma, bronchitis, atypical severe acute respiratory syndrome (SARS), pulmonary emphysema, etc. has become one of the health issues that have received great attentions worldwide. Acute lung injury (ALI) is a common and serious inflammatory lung disease with high death rate. Infectious pneumonia and sepsis are the main factors that cause ALI. Although new medical technology has been developed for control of ALI and the worse ARDS (acute respiratory distress syndrome), the mortality of ALI is still over 30%. The high mortality of these lung diseases can be reduced only when infection and inflammation are under control.

Refer to U.S. Pat. No. 8,591,896B2, a kind of chemokine receptor binding polypeptides specifically binding to chemokine receptor CXCR2 is revealed. The polypeptides are used for treatment of diseases involving aberrant functioning of CXCR2 such as cystic fibrosis, asthma, severe asthma, exacerbation of asthma, allergic asthma, acute lung injury, acute respiratory distress syndrome, idiopathic pulmonary fibrosis, airway remodeling, bronchiolitis obliterans syndrome, bronchopulmonary dysplasis, etc. However, the method for preparing polypeptides is complicated and costly with limited effect.

Kirenol is a biologically active substance isolated from plants such as Herba Siegesbeckiae, Siegesbeckia Glabrescens, etc. Previous studies demonstrate that kirenol has anti-arthritic and immune regulation activities. Refer to Chinese Pat. Pub. No. CN105012279B, a composition containing kirenol used in preparation of drugs for treatment of gouty arthritis, and rheumatoid arthritis (RA) is revealed. The composition is formed by kirenol and darutigenol. Refer to Chinese Pat. Pub. No. CN101880217B, kirenol used for treatment of autoimmune diseases and in preparation of immunosuppressants for prevention of transplanted organ rejection, and a method for extracting the same are revealed are disclosed.

There is no effective method used for treatment of ALI available now. Thus there is room for improvement and there is a need to find out various kinds of active substances used for treatment of ALI.

### SUMMARY OF THE INVENTION

Therefore it is a primary object of the present invention to provide a method for treatment of acute lung injury by use of kirenol. An effective does of kirenol is applied to an individual in need for reducing damages caused by acute lung injury (ALI).

Preferably, kirenol reduces hyaline membrane formation in lung, neutrophil infiltration in lung, and pulmonary edema.

Preferably, kirenol further reduces lipid peroxidation in lung and increases activities of antioxidant enzymes in lung such as superoxide dismutase, catalase and glutathione peroxidase.

Preferably, the effective dose of kirenol is between 3 mg/kg and 30 mg/kg.

Thereby Kirenol can be used in preparation of pharmaceutical compositions or food compositions for treatment of acute lung injury.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 shows sections of lung showing the effect of kirenol on hyaline membrane formation in lung of an embodiment according to the present invention;

FIG. 2 is a bar chart showing inhibition effect of kirenol on lung edema of an embodiment according to the present invention;

FIG. 3 is a bar chart showing the effect of kirenol on reduction of protein content in bronchoalveolar lavage fluid (BALF) of an embodiment according to the present invention;

FIG. 4 is a bar chart showing the effect of kirenol on reduction of the number of neutrophils in lung of an embodiment according to the present invention;

FIG. 5 is a bar chart showing the effect of kirenol on reduction of lipid peroxidation in lung of an embodiment according to the present invention;

FIG. 6 is a bar chart showing the effect of kirenol on increasing superoxide dismutase activity of an embodiment according to the present invention;

FIG. 7 is a bar chart showing the effect of kirenol on increasing catalase activity of an embodiment according to the present invention;

FIG. 8 is a bar chart showing the effect of kirenol on increasing glutathione peroxidase activity of an embodiment according to the present invention.

### DETAILED DESCRIPTION OF THE PREFERRED EMBODIMENT

In order to learn functions and features of the present invention, please refer to the following embodiments and related description in details.

The present invention provides a method for treatment of acute lung injury by use of kirenol. An effective dose of kirenol (3-30 mg/kg) is applied to an individual in need for treatment of acute lung injury (ALI). Kirenol not only reduces hyaline membrane formation, neutrophil infiltration, pulmonary edema, and lipid peroxidation in lung, but also increases activity of antioxidant enzymes in lung such as superoxide dismutase, catalase, and glutathione peroxidase.

Please refer to the following embodiments in order to learn applications of the present invention.

### Preparation: Establishment of Animal Models of ALI (Acute Lung Injury)

BALB/c mice (5-7 weeks old) are obtained from the National Laboratory Animal Center (NLAC) and maintained in the laboratory for a week in order to adapt to the environment. Then experiments are carried out as follows. The mice are divided into three groups. The control group: mice in the group are injected with normal saline by intratracheal injection (IT). The LPS stimulated group: mice in the group are treated with a 50 μL solution containing 100 μg of lipopolysaccharide (LPS) by IT for inducing ALI. The kirenol group: mice are given an intraperitoneal injection (IP) of 3 mg/kg, 10 mg/kg, and 30 mg/kg of kirenol respectively. After 30 minutes, each mouse is treated with 100 μg of LPS by IT. The dexamethasone group: mice are dosed with 1 mg/kg of dexamethasone by IP and then treated with 100 μg of LPS by IT after 30 minutes.

The mice in the respective group are killed 6 hours after LPS administration. Then use bronchoalveolar lavage (BAL) to get (Bronchoalveolar lavage fluid (BALF). And perform differential blood count, detect neutrophil activity and measure protein content in BALF. The lung tissue of the mice is used in histopathology examination, analysis of pulmonary edema, lipid peroxidation assay, and antioxidant activity analysis.

### Embodiment 1: Effect of Kirenol on Hyaline Membrane Formation in Lung

Kirenol used is obtained from ChemFaces (Catalog No. CFN98867) dissolved in Dimethyl sulfoxide (DMSO). While in use, Kirenol is diluted in DMSO to the concentration required.

A mouse lung lobe is treated by fixation, embedding, section, and dewaxing. After Hematoxylin & Eosin stain, the lung tissue is observed and accessed. Protein like fluid is leaked into and aggregated in alveoli to form hyaline membrane and cause noncardiogenic pulmonary edema when the alveolar capillary permeability is increased. At the moment, inflammatory cells proliferate in the lung. Thus hyaline membrane formation can be used as an indicator of the increased alveolar capillary permeability.

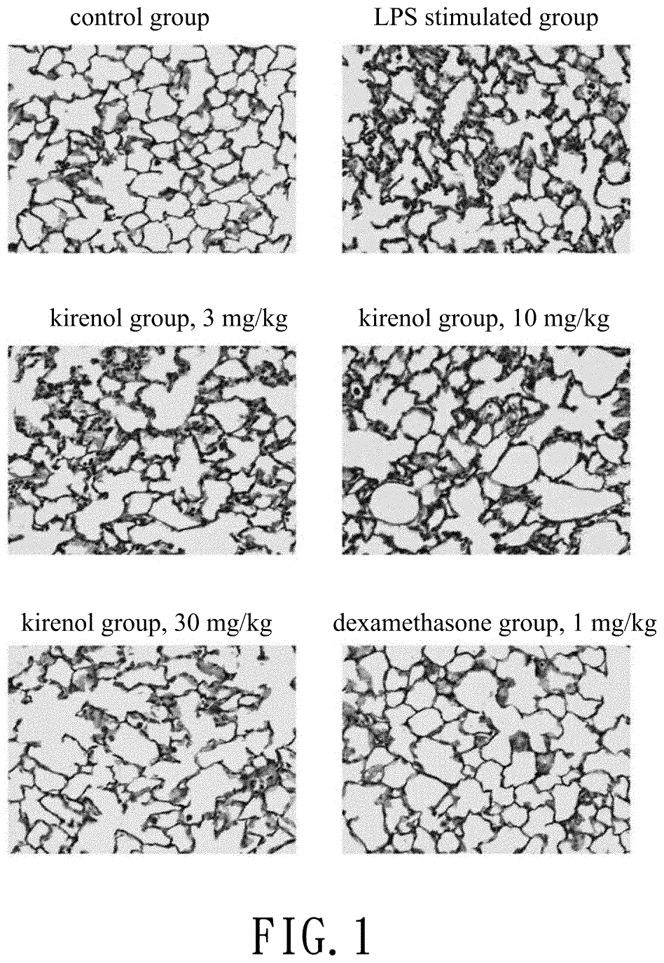

Refer to FIG. 1, histologically there is more hyaline membrane formation and more immune cell infiltration is observed in the LPS stimulated group compared with the control group. Kirenol reduces hyaline membrane formation in lung and immune cell infiltration induced by LPS in a dose dependent manner. As to the dexamethasone group, hyaline membrane formation in lung and immune cell infiltration are also significantly improved.

### Embodiment 2: Effect of Kirenol on Lung Tissue Wet/Dry Ratio

The mice are killed and lung tissue is obtained. The lung tissue is weighted to get wet weight. Then the lung tissue is dried in a 80° C. oven for 48 hr and re-weighted as dry weight. Thereby the wet/dry ratio of the lung tissue is calculated by dividing the wet weight by the dry weight.

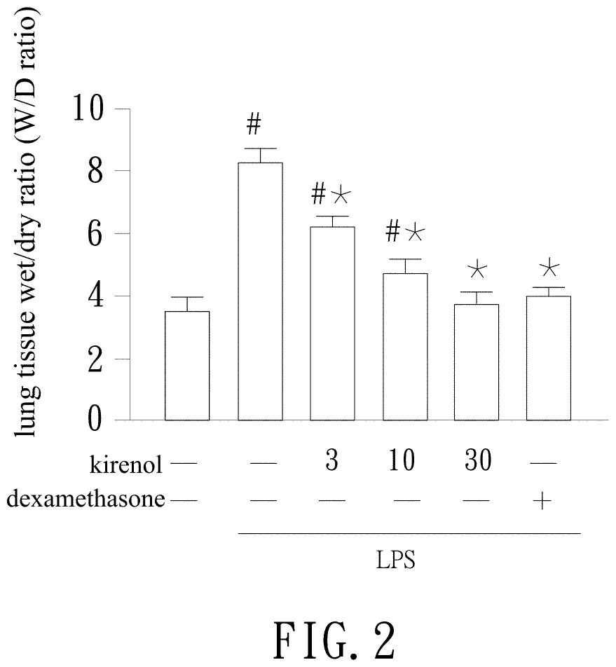

Refer to FIG. 2, the wet/dry ratio of the LPS stimulated group is clearly higher than the control group and this means mice in the LPS stimulated group have apparent lung edema. The wet/dry ratio of mice in the kirenol group treated with 3 mg/kg, 10 mg/kg, and 30 mg/kg of kirenol is reduced. The figure indicates that kirenol improves lung edema induced by LPS in a dose dependent fashion. The improvement of lung edema in the 30 mg/kg kirenol group is similar to that in the dexamethasone group. The results mentioned above are expressed as mean±SD (standard deviation) (n=5). “#” represents p<0.05 compared with the control group while “*” represents p<0.05 compared with the LPS stimulated group.

### Embodiment 3: Effect of Kirenol on Integrity of Alveolar Capillary Barrier

Polymorphonuclear leucocytes and protein in the capillary flow into the alveoli while alveolar capillary barrier being damaged. Thus the integrity of alveolar capillary barrier can be evaluated by analysis of the content of polymorphonuclear leucocytes and protein in the BALF. The test process is as follows. The mice are killed and the lung tissue is washed with 1×PBS buffer. After the lavage fluid being collected and centrifuged, measure protein content of supernatant by Bradford assay. As to polymorphonuclear neutrophils (PMN) precipitated in the bottom, they are fixed and stained with Giemsa stain (10% stain working solution). Then the number or the types of the PMN are observed and examined under a microscope.

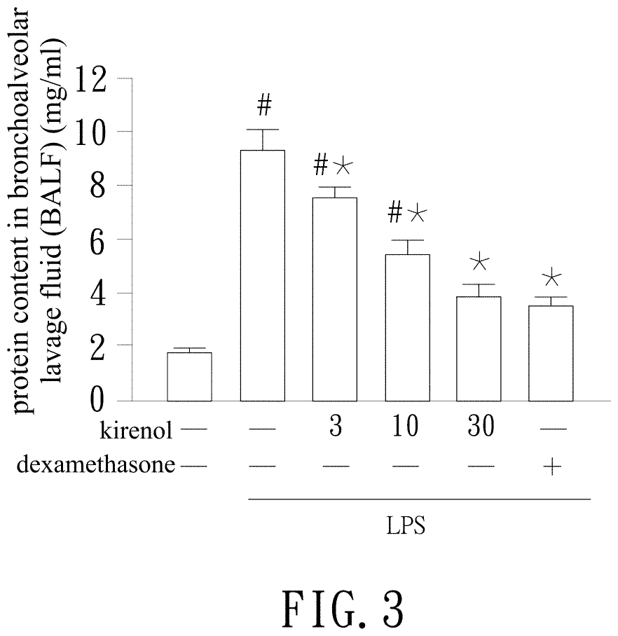

Refer to FIG. 3, the BALF of the LPS stimulated group has a higher protein content compared with the control group. As to the respective group treated with different doses of kirenol, the protein content is lowered. The figure demonstrates that kirenol prevents damage to the integrity of alveolar capillary barrier caused by LPS in a dose dependent way. The improvement of the integrity in the 30 mg/kg kirenol group is similar to that in the dexamethasone group. The above results are presented as mean±SD (n=5). “#” represents p<0.05 compared with the control group while “*” represents p<0.05 compared with the LPS stimulated group.

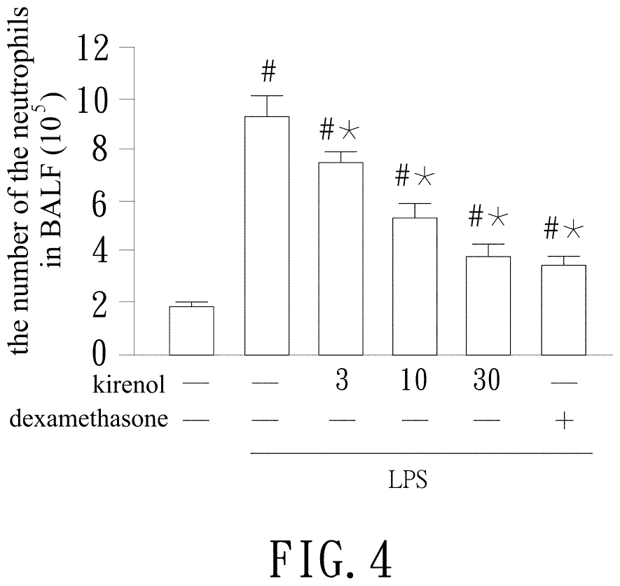

Refer to FIG. 4, there is much more neutrophils infiltrated in the lung of the mice in the LPS stimulated group compared with the control group. As to the group treated with different doses of kirenol, the number of the neutrophils is reduced. The figure shows that kirenol reduces neutrophil infiltration in alveolar cavity caused by LPS in a dose dependent manner. The number of the neutrophils in BALF of the 30 mg/kg kirenol group is similar to that of the dexamethasone group. The above results are presented as mean±SD (n=5). “#” represents p<0.05 compared with the control group while “*” represents p<0.05 compared with the LPS stimulated group.

### Embodiment 4: Effect of Kirenol on Lipid Peroxidation in Lung

For evaluation of lung tissue damage, the amount of malondialdehyde (MDA), the final product of lipid peroxidation in lung, is measured. The test process is as follows. The mice are killed and the lung tissue obtained is homogenized. Then the lung tissue is added with acetic acid, sodium dodecyl sulfate, and thiobarbituric acid, mixed evenly and heated up to 95° C. The mixed solution is cooled down to room temperature, added with n-butanol and pyridine, mixed and centrifuged. Lastly absorbance of the mixed solution is measured at 532 nm.

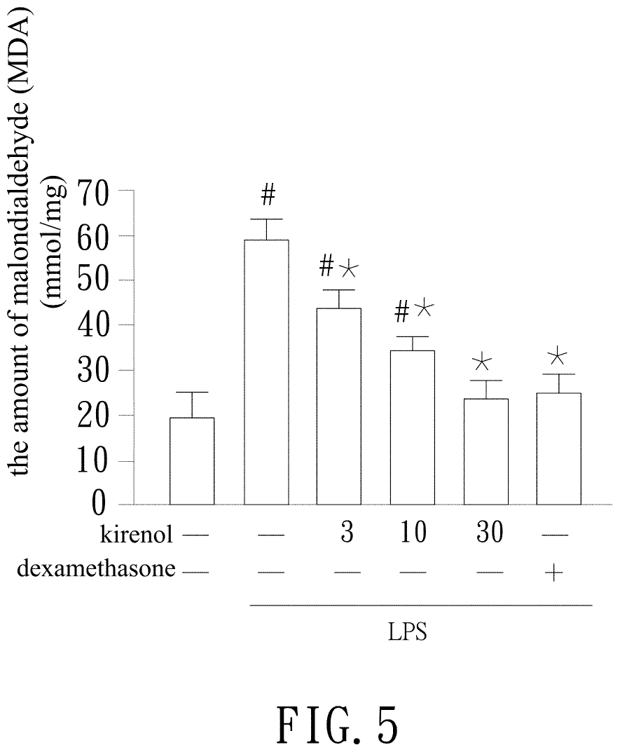

Refer to FIG. 5, lipid peroxidation of the LPS stimulated group is apparently higher than the control group while the respective group treated with kirenol has a lower lipid peroxidation. The figure demonstrates that kirenol prevents lipid peroxidation caused by LPS in a dose dependent manner. The inhibition effect of the 30 mg/kg kirenol group on lipid peroxidation is similar to that of the dexamethasone group. The above results are expressed as mean±SD (n=5). “#” represents p<0.05 compared with the control group while “*” represents p<0.05 compared with the LPS stimulated group.

### Embodiment 5: Effect of Kirenol on Activities of Antioxidant Enzymes in Lung

During acute lung injury, the increasing pulmonary oxidative stress easily leads to lung tissue damage. Under such condition, antioxidant enzymes in organisms can protect lung tissue against oxidants. For example, superoxide dismutase (SOD) is an enzyme that catalyzes the partitioning of superoxide radicals into hydrogen peroxide (H₂O₂) while catalase (CAT) and glutathione peroxidase (GSH) convert hydrogen peroxide into harmless water. In this embodiment, the mice lung tissue is dissolved and homogenized in solutions. Then measure activity of the respective antioxidant enzyme in supernatant after centrifugation.

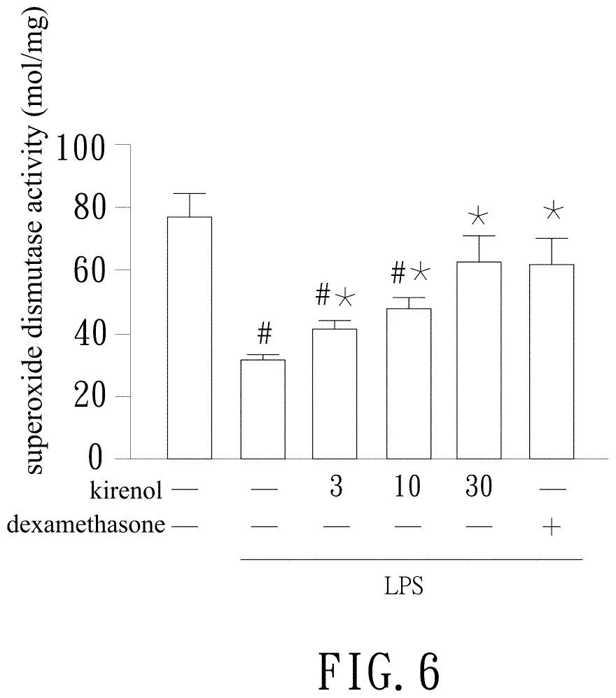

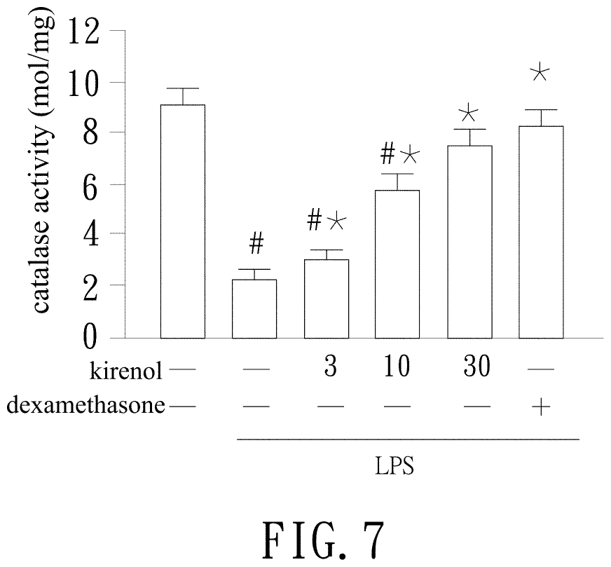

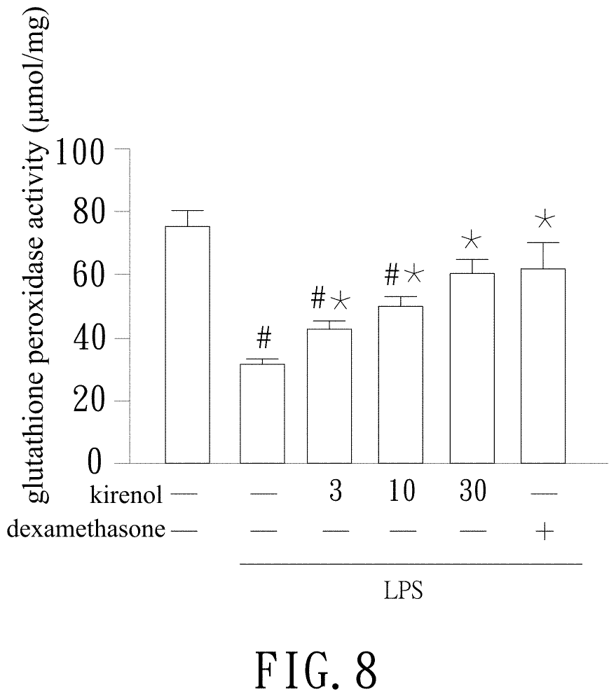

Refer to FIG. 6, FIG. 7 and FIG. 8, the activities of SOD, CAT, and GSH in the LPS stimulated group are all obviously decreased compared with the control group yet activities of the above three antioxidant enzymes in the groups treated with kirenol are increased in a dose dependent manner. The figures show that kirenol prevents reduction of antioxidant activities caused by LPS and the effect of the 30 mg/kg kirenol group on improving antioxidant activities is similar to that of the dexamethasone group. The results mentioned above are presented as mean±SD (n=5). “#” represents p<0.05 compared with the control group while “*” represents p<0.05 compared with the LPS stimulated group.

The present invention has the following advantages compared with the techniques available now:
1. Kirenol of the present invention not only reduces lung damage caused by acute lung injury effectively, but also prevents the increase of capillary permeability for maintaining integrity of alveolar capillary.
2. Kirenol with excellent antioxidant activity of the present invention further reduces lipid peroxidation and increases activities of antioxidant enzymes in lung in a dose dependent manner.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

1. A method for treatment of acute lung injury by use of kirenol comprising the step of:
applying an effective dose of kirenol in a range between 3 mg and 30 mg per 1 kg of a patient's weight to the patient in need for treatment of acute lung injury.

2. The method as claimed in claim 1, wherein the kirenol reduces hyaline membrane formation in lung, neutrophil infiltration in lung, and pulmonary edema.

3. The method as claimed in claim 1, wherein the kirenol further reduces lipid peroxidation and increases activities of antioxidant enzymes in lung.

4. The method as claimed in claim 3, wherein the antioxidant enzymes include superoxide dismutase, catalase and glutathione peroxidase.

5. (canceled)
